(19) United States
(12) Patent Application Publication
(10) Pub. No.: US 2026/0284035 A1
(43) Pub. Date: Sep. 24, 2026

(54) COMBINATIONS INCLUDING METHOCINNAMOX

(71) Applicant: BOARD OF REGENTS, THE UNIVERSITY OF TEXAS SYSTEM, Austin, TX (US)

(72) Inventor: Charles Patrick FRANCE, Austin, TX (US)

(21) Appl. No.: 19/472,198

(22) PCT Filed: Apr. 4, 2024
(86) PCT No.: PCT/US2024/022939

§ 371 Date: Oct. 3, 2025

Related U.S. Application Data
(60) Provisional application No. 63/494,024, filed on Apr. 4, 2023.

(51) Int. Cl. A61K 31/485; A61K 31/145; A61K 31/185; A61K 31/195; A61K 31/197; A61K 31/357; A61P 25/36

(52) U.S. Cl. CPC A61K 31/485; A61K 31/145; A61K 31/185; A61K 31/195; A61K 31/197; A61K 31/357; A61P 25/36

(57) ABSTRACT

Disclosed herein are compositions and methods for treating opioid use disorders, including opioid overdose with combinations including methocinnamox and at least one additional opioid modulators, for example opioid agonists, partial agonists, antagonists and allosteric modulators. The compositions and methods are also useful for treating alcohol use disorder, and other disorders associated with endogenous opioid production.

### CROSS-REFERENCE TO RELATED APPLICATION

This application claims the benefit of U.S. Provisional Application 63/494,024, filed Apr. 4, 2023, the contents of which is hereby incorporated in its entirety.

### STATEMENT OF GOVERNMENT SUPPORT

This invention was made with government support under R01DA048417 and UG3DA048387, awarded by the National Institute of Drug Abuse, National Institutes of Health. The government has certain rights in the invention.

### FIELD OF THE INVENTION

The invention relates to methocinnamox in combination with one or more opioid receptor modulators, for example opioid agonists, partial agonists, antagonists and allosteric modulators. The combinations are useful for treatment of opioid and alcohol use disorders.

### BACKGROUND

Opioids, including morphine, fentanyl, codeine, and oxycodone, have an essential medical role both as analgesics and anesthetics. Opioids reduce perception of pain by binding to opioid receptors in the central nervous system. These receptors include mu, kappa, sigma, delta, and epsilon subtypes. Mu and sigma receptors activate the dopaminergic mesolimbic system, triggering the release of dopamine and creating a feeling of euphoria and well-being. Nevertheless, opioids are also highly susceptible to abuse and are considered highly addictive. Opioid Use Disorder (OUD) is a neurological condition that arises as the consequence of repeated and compulsive use of opioid drugs. With prolonged use, opioids can cause several negative signs and symptoms besides addiction, including negative mood, muscle cramps, diarrhea, anxiety, and tremors. Additionally, as opioid use continues, the brain adapts to the drugs, causing increased tolerance and leading subjects to take higher doses of the drug. This, in turn, increases the risk of opioid overdose and death. Opioid use disorder, including overdose, can arise when opioids are used recreationally, i.e., non-medically prescribed opioid use disorder, and also when the opioids are used (at first) for a legitimate medical use which then leads to consumption of the drugs in non-prescribed doses (prescribed opioid use disorder).

Opioid overdose is a significant problem throughout the world; it affects recreational drug users and as well as individuals who abuse drugs, as well as patients treated with prescribed medications. Overdose deaths from fentanyl, heroin and other opioids have reached alarming numbers. In the United States, data from the National Center for Health Statistics showed a steady increase from 15,800 deaths in 2005, to 40,100 deaths in 2017. Drug overdose now accounts for over 100,000 deaths annually, most involving opioids, and the number of deaths is constantly increasing. Opioid intoxication is manifested by reduced consciousness and respiratory depression which may deteriorate to cardiac arrest and death. Overdose of opioids leads to depressed heart rate and breathing, leading to hypoxia. Hypoxia leads to short- and long-term effects on the central nervous system, including coma and permanent brain damage, which can result in the death of the hypoxic person. Overdoses of opioids, particularly heroin and fentanyl are very common. Fentanyl is a synthetic compound that is much more potent that morphine. Fentanyl analogs, which can be even more potent that fentanyl, further increase the risk of overdose if a user is unfamiliar with the strength of a particular analog in comparison with regular fentanyl or morphine. It is not unheard of for people to overdose the very first time they use powerful opioids. These risks are compounded when opioids are taken in combination with other substances, including alcohol or benzodiazepines.

In view of the potentially tragic outcome of overdose, a subject that has overdosed on an opioid requires urgent medical attention. The only medicines that can be employed to treat opioid overdoses effectively are opioid receptor antagonists, which act by binding to opioid receptors, displacing opioid agonists (like heroin) without eliciting opioid effects of their own, whether intended (e.g. euphoria) or unintended and/or potentially dangerous (including respiratory depression). Emergency administration of opioid antagonists can reduce (sometimes completely) the degree of opioid intoxication and, in essence, ‘reverse’ an opioid overdose.

The opioid antagonist naloxone can be used to treat overdose, and is available in the form of a single dose either as a liquid nasal spray (Narcan®; which is sprayed directly into one nostril), or as an auto-injector (Evzio®; which delivers drug by injection into the muscle or under the skin). These products are undoubtedly effective in helping to save lives. However, naloxone has a relatively short half-life. The short half-life can lead to re-narcotization, in which naloxone is cleared from the rescued victim while dangerous levels of opioids remain. In such an event, opioid intoxication symptoms can re-emerge, leading to cardiac arrest and death. As such, in about one third of overdose cases, two or more doses administered over time are needed to completely protect a subject from the harm of the overdose.

Beyond death and other medical catastrophes, chronic substance abuse places enormous burdens on society. Efforts to reduce the numbers of people that abuse alcohol and controlled substances such as opioids have been continuous and relatively unsuccessful. There is a relationship between alcohol intake and endogenous opioid production. Ethanol intake increases the synthesis and release of endogenous opioids (i.e., endorphins), which contributes to the positive reinforcement properties of ethanol. These findings have suggested the use of opioid receptor antagonists (such as naloxone or naltrexone) to prevent ethanol-induced analgesia, intoxication, and coma. Opioid antagonists, such as naltrexone, have been used to reduce the rate of relapse to heavy drinking in alcohol dependent patients.

Methocinnamox (“MCAM”) is long-acting and highly selective opioid receptor antagonist.

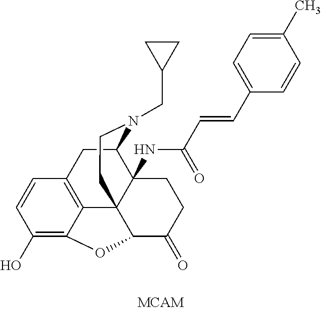 [SMILES: Cc1ccc(/C=C/C(=O)N[C@@]23CCC(=O)[C@@H]4Oc5c(O)ccc6c5[C@@]42CCN(CC2CC2)[C@@H]3C6)cc1]

Compared with other opioid receptor antagonists, MCAM binds to opioid receptors extended periods of time, so that its effects are very long lasting compared with other antagonists. As such MCAM is an effective medication for treating opioid abuse, because of its long duration of action. A subject receiving MCAM is less likely to seek out opioids, because the MCAM effectively renders the subject immune to the euphoric effects of the opioid. MCAM is also useful for treating opioid overdose, because of its long duration of protection. Moreover, the risk of re-narcotization is much less likely when overdose is treated with MCAM than other antagonists.

There remains a need for improved therapeutic compositions and methods for the treatment and prevention of opioid use disorder. There remains a need for improved therapeutic compositions and methods for the treatment and prevention of substance abuse, including alcohol use disorder. There remains a need for improved therapeutic compositions and methods for the treatment and prevention of opioid overdose.

### BRIEF DESCRIPTION OF THE FIGURES

FIG. 1 depicts how mixtures of MCAM and naltrexone completely blocked the effects of a very large dose of fentanyl. Monkeys received MCAM daily and with increasing dose, the fentanyl dose-response curve was shifted progressively rightward. However, when the dose of MCAM was increased from 0.1 to 0.32 mg/kg/day, the fentanyl dose-response curve was not shifted further to the right (FIG. 1A). When a small dose of the opioid receptor antagonist naltrexone (0.1 mg/kg) was administered along with the daily dose of 0.32 mg/kg MCAM, the effects of 32 μg/kg/infusion fentanyl were blocked completely (FIG. 1B). In sessions when monkeys self-administered a unit dose of 100 μg/kg/infusion fentanyl, they received 3 times the lethal dose of fentanyl in the 90-minute session—with no adverse effect and no apparent change in behavior.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Compounds disclosed herein may be provided in the form of pharmaceutically acceptable salts. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitratc.

In certain implementations, the methocinnamox is formulated as the hydrochloride salt. In some implementations the methocinnamox is provided as a salt with an organic acid (for instance an alkyl carboxylic acid). In some implementations, the methocinnamox is formulated as the maleate salt. In some implementations, the methocinnamox is formulated as the malate salt. In some implementations, the methocinnamox is formulated as the lactate salt.

It has been surprisingly discovered that methocinnamox, when used in combination with one or more additional opioid receptor modulators, can exert synergistic pharmacologic effects, for instance in terms of duration and/or antagonistic effect. As such, the disclosed combinations can be used in reduced amounts, and/or with reduced dosing frequencies, relative to when the methocinnamox or antagonist is administered separately.

As used herein, “opioid overdose” refers to instances in which a subject receives larger quantities of as opioid than may be safely taken. Symptoms arising from opioid overdose can include central nervous system and respiratory depression, hypoxia, miosis, and apnea, any one of which lead to death if not treated rapidly. As such, disclosed herein are methods of treating an overdose in a subject by administering the disclosed combinations to a patient suffering an overdose. The disclosed combinations can be used to prevent overdose, for example in cases of accidental exposure in a medical or criminal setting. The disclosed combinations can be used prophylactically, for instance if a subject is at risk of being exposed to unsafe levels of opioids (for example an aerosolized opioid chemical agent).

As used herein, a subject refers to an organism receiving the disclosed combinations. In some implementations the subject is a human, for example an adult human, a pediatric human (less than 18 years of age), or a geriatric human (greater than 64 years of age). In some implementations the subject is a domestic animal, for instance a companion animal like a dog or cat, a livestock animal like a cow or pig, a working animal like a horse or donkey, or an exotic animal like an elephant, lion, or tiger.

The disclosed combinations may also be used in the treatment of conditions mediated by endogenous opioid agonists (e.g. endorphins), which conditions may be collectively classified together as “endorphin-mediated hedonia,” including but not limited to addictive behaviors like excessive eating (bulimia), drinking (alcoholism), exercise, sex, gambling, etc.

The invention is also directed to a method for treating drug and/or alcohol addiction (involving drug dependency or drug abuse) during withdrawal therapy by administering the disclosed combinations to a patient at a dosage sufficient to reduce or eliminate one or more symptoms associated with withdrawal. Such symptoms include nausea, vomiting, anxiety, abdominal cramps, muscle pain, chills and headache. In addition, the combinations can decrease the drug cravings typically experienced by addicts after cessation of the abused substance. The disclosed combinations are especially useful in the treatment of opioid use disorder such as heroin, morphine, fentanyl, oxycodone, oxymorphone, and methadone (among others). However, they can also be used in treating patients addicted to cocaine, alcohol, amphetamines and combinations of these drugs.

Disclosed herein are methods of treating an opioid use disorder in a subject, by administering to the subject an effective amount of methocinnamox, or a pharmaceutically acceptable salt thereof, in combination with at least one additional opioid receptor modulator.

In certain implementations the combinations can be used for treating or preventing opioid dependence. In certain implementations the combinations can be used for reducing or preventing opioid withdrawal symptoms. In some implementations, the combinations can be used for reducing opioid cravings. In some implementations the combinations can be used for treating or preventing opioid overdose, including in some implementations for treating or preventing opioid-associated respiratory depression. In some implementations, the combinations can be used for reducing and/or preventing opioid poisoning. In certain implementations the disclosed combinations can be used to treat or prevent opioid addiction. In certain implementations, the disclosed combinations can be used to prevent relapse in a recovering addict; by blocking the receptors targeted by opioids, the subject does not experience the euphoria upon resumption of opioid use and is thus better able to maintain abstinence from the opioid.

In some implementations the disclosed combinations can be used for reducing or preventing one or more signs or symptoms of opioid toxicity. Exemplary symptoms of opioid toxicity include respiratory depression or ventilatory depression, hypoxia, loss of consciousness, decreased respiratory rate, decreased respiratory depth, apnea, delirium, hypotension, bradycardia, decreased body temperature, urinary retention, pupil miosis, sedation, dizziness, nausea, vomiting, constipation, hyperalgesia, immunologic and hormonal dysfunction, muscle rigidity, myoclonus, and bowel dysfunction.

In some implementations the combinations can be used for reducing or preventing renarconization in a patient being treated for opioid overdose. In some implementations, the combinations can be used for treating or preventing opioid overdose, including opioid-associated respiratory depression, by administering a single dose of the disclosed combinations.

In some implementations, the combinations can be used for preventing opioid dependence in a patient receiving an opioid analgesic, and in some implementations the combinations can be used for treating or preventing an opioid-induced adverse event, including opioid overdose, in a patient receiving an opioid analgesic. In some implementations the opioid-induced adverse effect includes one or more of bowel dysfunction, nausea, vomiting, somnolence, dizziness, respiratory depression, headache, dry mouth, sedation, sweats, asthenia, hypotension, dysphoria, delirium, miosis, pruritis, urticaria, urinary retention, hyperalgesia, allodynia, physical dependence, or tolerance. In certain implementations, the combinations can be used for reducing and/or preventing opioid-induced hypoxia.

The disclosed combinations can be used in to treat or prevent overdose of any opioid, and in some implementations the combinations can be used to treat fentanyl and/or fentanyl analog overdose. Fentanyl analogs are structurally related to fentanyl, with one or more modifications affecting one of more physical and/or pharmacological properties of the drug. Fentanyl analogs are also created in order to avoid detection and scheduling from regulatory and law enforcement regimes. In certain implementations, the fentanyl analog has the formula:

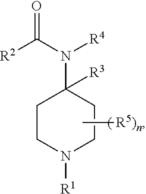

wherein
R¹ is H or C₁₋₆alkyl, optional substituted one or more times by OH, CO₂C₁₋₆alkyl, aryl, C₃₋₁₀cycloalkyl, C₂₋₁₀heterocyclyl, or C₁₋₁₀heteroaryl;
R² is C₁₋₆alkyl, C₂₋₄alkenyl, C₃₋₁₀cycloalkyl or C₁₋₁₀heteroaryl; wherein R² may be substituted by aryl, OC₁₋₆alkyl;
R³ is H, CO₂C₁₋₆alkyl, C₁₋₆alkyl, aryl, wherein R³ may be substituted by OC₁₋₆alkyl;
R⁴ is aryl or C₁₋₁₀heteroaryl, wherein R⁴ substituted one or more times by F;
R⁵ is in each case independently selected from C₁₋₆alkyl, C₂₋₄alkenyl, and F; and
n is 0, 1, or 2.

In some embodiments, the disclosed combinations can be used to treat or prevent overdose of a fentanyl analog such as sufentanil, alfentanil, remifentanil, carfentanil, furanylfentanyl, 4-fluorobutyrylfentanyl, 4-methoxyburtyrylfentanyl, acrylfentanyl, 4-chloroisobutyryfentanyl (4Cl-iBF), 4-fluoroisobutyrfentanyl (4F-iBF), tetrahydrofuranfentanyl (THF-F), cyclopentylfentanyl, AH-7921, U-47700, MT-45, AH-7921, U-47700, AH-7921, or a combination thereof.

The disclosed combinations are also useful to treat or prevent overdoses of other opioids, for instance morphine overdose, heroin overdose, oxycodone overdose, hydrocodone overdose, oxymorphone overdose, hydromorphone overdose, codeine overdose, dihydrocodeine overdose, tramadol overdose, buprenorphine overdose, methadone overdose, etorphine overdose or a combination thereof.

The disclosed combinations are also used to treat or prevent overdoses with concomitant use of alcohol, barbiturates, xylazine, benzodiazepines, cocaine, amphetamines, gamma-hydroxy butyrate, PCP, ketamine, or a combination thereof.

The disclosed combinations can also be used to treat or prevent an alcohol use disorder in a subject, for example to treat or prevent alcohol dependence, to assist with alcohol detoxification, to reduce alcohol cravings, and to treat or prevent alcohol overdose.

The disclosed combinations can be used to reduce or prevent alcohol use disorder in an at-risk patient. At-risk patients include those with risk factors that increase the likelihood that alcohol dependence could occur. In some implementations, the at-risk patient is a trauma-survivor. In some implementations the at-risk patient has a family history of alcohol use disorder and/or a personal history of early-onset alcohol use. In some implementations the at-risk patient has received bariatric surgery. In some implementations the at-risk patient has a history of continuous alcohol use that does not rise to the clinical definition of alcohol use disorder.

The disclosed combinations can also be used in the long-term treatment of alcohol use disorder. For example to disclosed combinations can be used, alone or in combination with supportive counseling, to reduce the level of alcohol consumption in a subject. The disclosed combinations can be used, alone or in combination with supportive counseling, in an alcohol-cessation program. The disclosed combinations can be administered in conjunctive with other therapeutic agents for alcohol use disorder, including alcohol dehydrogenase inhibitors like disulfiram, NMDA antagonists like acamprosate, or anticonvulsants like gabapentin, pregabalin, or topiramate. The disclosed combinations may be used in combinations with antidepressants and other serotonergic agents like odansetron, tropisetron, granisetron, dolasetron, palonosetron, or ramosetron.

In certain implementations, the disclosed combinations include methocinnamox, or salt thereof, with one or more of naltrexone, naloxone, nalmefene, diprenorphine, nalorphine, nalorphine dinicotinate, levallorphan, samidorphan, nalodiene, buprenorphine, dezocine, eptazocine, butorphanol, levorphanol, nalbuphine, pentazocine, phenazocine, cyprodime, naltrindole, norbinaltorphimine, salts thereof. In certain implementations the disclosed combinations include methocinnamox (or salt thereof) and naltrexone (e.g., naltrexone HCl). In some implementations the disclosed combinations include methocinnamox (or salt thereof) and naloxone (e.g., naloxone HCl). In further implementations, the disclosed combinations include methocinnamox (or salt thereof), naltrexone (e.g., naltrexone HCl), and naloxone (e.g., naloxone HCl).

In some implementations, the methocinnamox, or salt thereof, can be administered in a composition containing methocinnamox in an amount of 0.5-500 mg, 0.5-250 mg, 0.5-100 mg, 0.5-50 mg, 0.5-25 mg, 0.5-20 mg, 0.5-15 mg, 0.5-10 mg, 0.5-5 mg, 0.5-2.5 mg, 1-2.5 mg, 1-5 mg, 1-10 mg, 2.5-7.5 mg, 5-15 mg, 10-20 mg, 15-25 mg, 20-30 mg, 25-50 mg, 25-75 mg, 50-100 mg, 100-250 mg, or 250-500 mg, wherein the amount is measured as the free base equivalent. In the context of human subjects, the methocinnamox (or salt thereof) can be administered at a dose of 0.1-10 mg/kg, 0.1-8 mg/kg, 0.1-6 mg/kg, 0.1-5 mg/kg, 0.1-4 mg/kg, 0.1-3 mg/kg, 0.1-2 mg/kg, 0.1-1 mg/kg, 0.5-1.5 mg/kg, 1-2 mg/kg, 1.5-2.5 mg/kg, 2-5 mg/kg, 2.5-7.5 mg/kg, or 5-10 mg/kg (calculated as the free base).

In some implementations, the additional opioid receptor modulator, e.g., naltrexone (e.g., naltrexone HCl) or naloxone (e.g., naloxone HCl), can be administered in a composition containing additional opioid receptor modulator in an amount of 0.5-100 mg, 0.5-50 mg, 0.5-25 mg, 0.5-20 mg, 0.5-15 mg, 0.5-10 mg, 0.5-5 mg, 0.5-2.5 mg, 1-2.5 mg, 1-5 mg, 1-10 mg, 2.5-7.5 mg, 5-15 mg, 10-20 mg, 15-25 mg, 20-30 mg, 20-40 mg, 25-50 mg, 25-75 mg, or 50-100 mg, wherein the amount is measured as the free base equivalent. In certain implementations, the additional opioid receptor modulator, e.g., naltrexone (e.g., naltrexone HCl) or naloxone (e.g., naloxone HCl), can be administered to a human subject in a dose of 0.01-3 mg/kg, 0.01-2 mg/kg, 0.01-1 mg/kg, 0.05-1 mg/kg, 0.1-1 mg/kg, 0.1-0.5 mg/kg, 0.25-0.75 mg/kg, 0.5-1 mg/kg, 0.01-0.1 mg/kg, 0.05-0.1 mg/kg, 0.025-0.075 mg/kg, 0.01-0.05 mg/kg, or 0.075-0.125 mg/kg, wherein the amount is measured as the free base equivalent.

In some implementations, the methocinnamox (or salt thereof) and the additional opioid receptor modulator, e.g., naltrexone (e.g., naltrexone HCl) or naloxone (e.g., naloxone HCl), are administered in the same event dosing event. The agents can be administered in a unitary dosage form containing both active agents, or the agents can be administered simultaneously in two separate compositions. In some implementations the methocinnamox (or salt thereof) and the additional opioid receptor modulators can be administered within a period of 168 hours, 144 hours, 120 hours, 96 hours, 72 hours, 48 hours, 36 hours, 24, hours, 12 hours, 6 hour, 4 hours, 3 hours, 2 hours, 1 hour, 30 minutes, 15 minutes, 10 minutes, 5 minutes, or 2 minutes of each other. In some implementations, the methocinnamox (or salt thereof) is administered before the opioid receptor modulator, while in other implementations the methocinnamox (or salt thereof) is administered after the opioid receptor modulator.

In some implementations the methocinnamox and additional antagonist can be administered according to different dosing schedules. For example, methocinnamox can be administered once every 3 days, once every 5 days, once every 7 days, once every 10 days, or once every 14 days, while the additional antagonists is administered each day, once or twice a day. In some implementation the additional antagonist is provided as a depot formulation for continual release of the antagonist, and the methocinnamox is administered once every 3 days, once every 5 days, once every 7 days, once every 10 days, or once every 14 days. In certain implementations, the period between MCAM administration can be at least 3 days, at least 5 days, at last 7 days, at least 10 days, or at least 14 days.

The methocinnamox (or salt thereof) can be administered (either a single agent or in a dosage form containing more than one active ingredients) enterally, parenterally, intranasally, vaginally, by inhalation, or a combination thereof. When the methocinnamox is administered enterally, it may be administered by oral administration, sublingual administration, buccal administration, rectal administration, or a combination thereof. When the methocinnamox is administered parenterally, it may be administered by intramuscular injection, intravenous injection, subcutaneous injection, or a combination thereof.

In certain implementations, the methocinnamox (or salt thereof) is administered intranasally, either as a single agent or in a dosage form containing an additional antagonist. Naloxone is an especially preferred antagonist when methocinnamox is administered intranasally.

In certain implementations, the methocinnamox (or salt thereof) is administered by inhalation, either as a single agent or in a dosage form containing an additional antagonist. Naloxone is an especially preferred antagonist when methocinnamox is administered by inhalation.

For implementations when the additional opioid receptor modulator is administered separately from the methocinnamox, the additional opioid receptor modulator may be administered enterally, parenterally, intranasally, vaginally, by inhalation, or a combination thereof.

When the additional opioid receptor modulator is administered separately from methocinnamox and enterally, it may be administered by oral administration, sublingual administration, buccal administration, rectal administration, or a combination thereof. When the additional opioid receptor modulator is administered orally, it is especially preferred that the additional opioid receptor modulator is naltrexone (e.g., naltrexone HCl).

When the additional opioid receptor modulator is administered separately from methocinnamox and parenterally, it may be administered by intramuscular injection, intravenous injection, subcutaneous injection, or a combination thereof.

When the additional opioid receptor modulator is administered separately from methocinnamox and intranasally, it is especially preferred that the additional opioid receptor modulator is naloxone (e.g., naloxone HCl).

When the additional opioid receptor modulator is administered separately from methocinnamox and by inhalation, it is especially preferred that the additional opioid receptor modulator is naloxone (e.g. naloxone HCl).

Also disclosed herein are pharmaceutical compositions that include methocinnamox, or a pharmaceutically acceptable salt thereof, and at least one additional opioid receptor modulator.

In certain implementations, the pharmaceutical compositions include one or more opioid receptor modulator such as naltrexone, naloxonc, nalmefene, diprenorphinc, nalorphinc, nalorphine dinicotinate, levallorphan, samidorphan, nalodiene, buprenorphine, dezocine, eptazocine, butorphanol, levorphanol, nalbuphine, pentazocine, phenazocine, cyprodime, naltrindole, norbinaltorphimine, salts thereof, or a combination thereof. In certain implementations, the additional opioid receptor modulator is naltrexone (e.g., naltrexone HCl). In some implementations, the additional opioid receptor modulator is naloxone (e.g., naloxone HCl). In some implementations, the additional opioid receptor modulator is naloxone (e.g., naloxone HCl) and naltrexone (e.g., naltrexone HCl).

In some implementations the pharmaceutical composition includes methocinnamox, or a pharmaceutically acceptable salt thereof, in an amount of 0.5-500 mg, 0.5-250 mg, 0.5-100 mg, 0.5-50 mg, 0.5-25 mg, 0.5-20 mg, 0.5-15 mg, 0.5-10 mg, 0.5-5 mg, 0.5-2.5 mg, 1-2.5 mg, 1-5 mg, 1-10 mg, 2.5-7.5 mg, 5-15 mg, 10-20 mg, 15-25 mg, 20-30 mg, 25-50 mg, 25-75 mg, 50-100 mg, 100-250 mg, or 250-500 mg, wherein the amount is measured as the free base equivalent.

The pharmaceutical composition may include the additional opioid receptor modulator in an amount of 0.5-100 mg, 0.5-50 mg, 0.5-25 mg, 0.5-20 mg, 0.5-15 mg, 0.5-10 mg, 0.5-5 mg, 0.5-2.5 mg, 1-2.5 mg, 1-5 mg, 1-10 mg, 2.5-7.5 mg, 5-15 mg, 10-20 mg, 15-25 mg, 20-30 mg, 20-40 mg, 25-50 mg, 25-75 mg, or 50-100 mg, wherein the amount is measured as the free base equivalent.

Also disclosed herein are kits for the treatment of an opioid use disorder (as defined herein), or the treatment of an alcohol use disorder (as defined herein). The kits include comprising methocinnamox, or a pharmaceutically acceptable salt thereof, and at least one additional opioid receptor modulator. In some implementations, the kits include naltrexone, naloxone, nalmefene, diprenorphine, nalorphine, nalorphine dinicotinate, levallorphan, samidorphan, nalodiene, buprenorphine, dezocine, eptazocine, butorphanol, levorphanol, nalbuphine, pentazocine, phenazocine, cyprodime, naltrindole, norbinaltorphimine, salts thereof, or a combination thereof. Especially preferred kits include those containing methocinnamox with naltrexone (e.g., naltrexone HCl) and/or naloxone (e.g., naloxone HCl).

In some implementations, the kits contain methocinnamox, or a salt thereof, and the additional opioid receptor modulator is separate dosage forms. In some implementations, the kits include methocinnamox, or salt thereof, and additional opioid receptor modulator in the same unit dosage form. The kits may include a single dose of methocinnamox, or salt thereof, while in some implementations the kits include multiple doses of methocinnamox, or salt thereof.

### EXAMPLES

The following examples are for the purpose of illustration of the invention only and are not intended to limit the scope of the present invention in any manner whatsoever.

### Example 1

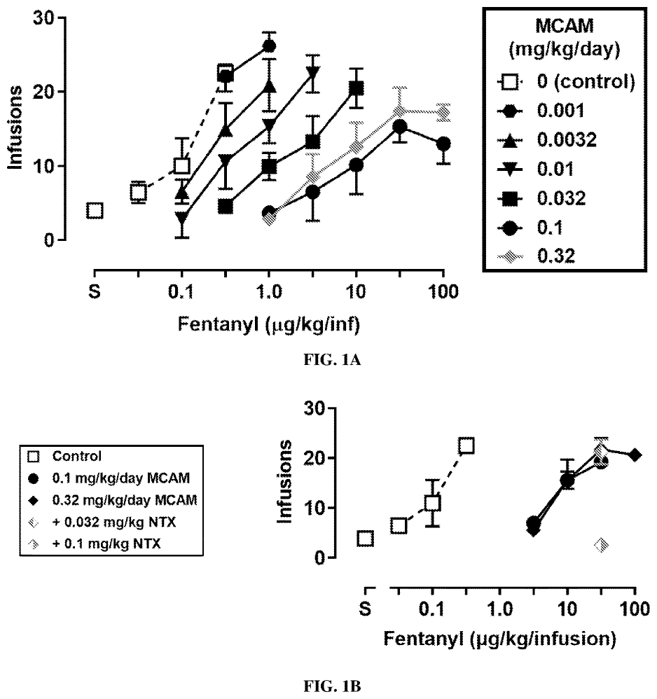

Rhesus monkeys were surgically instrumented with intravenous catheters and trained to press a lever in daily 90-minute sessions to receive an infusion of saline(S) or fentanyl. Different doses of fentanyl were studied on different days to construct a fentanyl dose-response curve (open squares, FIGS. 1A and 1B). Next, monkeys received a single subcutaneous injection of MCAM daily, beginning with a dose of 0.001 mg/kg/day and increasing across months to a maximum of 0.32 mg/kg/day. The smallest dose of MCAM did not affect responding for fentanyl; however, larger doses antagonized fentanyl as evidenced by a progressive, shift to the right and downward in the fentanyl dose-response curve (filled symbols, FIG. 1A). The shift to the right in the fentanyl dose-response curve was dose-related up to a dose of 0.1 mg/kg/day MCAM. A further increase in the dose of MCAM, to 0.32 mg/kg/day, did not shift the fentanyl dose-response curve further to the right or downward, suggesting that the magnitude of shift in the fentanyl curve was the maximal effect that could be obtained with MCAM. When monkeys that continued to receive 0.32 mg/kg/day MCAM also received an acute injection of 0.1 mg/kg naltrexone, monkeys did not respond for fentanyl, indicating that naltrexone blocked an effect of fentanyl that was no longer sensitive to antagonism by MCAM (FIG. 1B). Collectively, these results suggest that a combination of MCAM with another opioid receptor antagonist might provide the most effective method for blocking the effects of fentanyl and other opioid receptor agonists.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

What is claimed is:

1. A method of treating an opioid use disorder in a subject, comprising administering to the subject in need thereof an effective amount of methocinnamox, or a pharmaceutically acceptable salt thereof, in combination with at least one additional opioid receptor modulator.

2. The method according to claim 1, wherein the methocinnamox is methocinnamox HCl, methocinnamox malate, methocinnamox maleate, or methocinnamox lactate.

3. The method according to claim 1, wherein the methocinnamox is methocinnamox maleate.

4. The method according to any of claims 1-3, wherein the method is for treating or preventing opioid dependence.

5. The method according to any of claims 1-3, wherein the method is for reducing or preventing opioid withdrawal symptoms.

6. The method according to any of claims 1-3, wherein the method is for reducing opioid cravings.

7. The method according to any of claims 1-3, wherein the method is for treating or preventing opioid overdose.

8. The method according to any of claims 1-3, wherein the method is for treating or preventing opioid-associated respiratory depression.

9. The method according to any of claims 1-3, wherein the method is for reducing or preventing renarconization in a patient being treated for opioid overdose.

10. The method according to any of claims 1-3, wherein the method is for preventing opioid dependence in a patient receiving an opioid analgesic.

11. The method according to any of claims 1-3, wherein the method is for preventing opioid overdose in a patient receiving a medically prescribed opioid.

12. The method according to any of claims 1-3, wherein the method is for preventing an opioid-induced adverse event in a patient receiving a medically prescribed opioid.

13. The method according to any of claims 1-3, wherein the method is for preventing an opioid-induced adverse event in a patient receiving a medically prescribed opioid, wherein the opioid-induced adverse effect comprises bowel dysfunction, nausea, vomiting, somnolence, dizziness, respiratory depression, headache, dry mouth, sedation, sweats, asthenia, hypotension, dysphoria, delirium, miosis, pruritis, urticaria, urinary retention, hyperalgesia, allodynia, physical dependence, tolerance, or a combination thereof.

14. The method according to any of claims 1-3, comprising reducing and/or preventing opioid-induced hypoxia.

15. The method according to any of claims 1-3, comprising reducing and/or preventing opioid poisoning.

16. The method according to any of claims 1-3, wherein the opioid use disorder is fentanyl overdose, fentanyl analog overdose, morphine overdose, heroin overdose, oxycodone overdose, hydrocodone overdose, oxymorphone overdose, hydromorphone overdose, codeine overdose, dihydrocodeine overdose, tramadol overdose, buprenorphine overdose, methadone overdose, etorphine or a combination thereof.

17. The method according to claim 16, wherein the fentanyl analog has the structure: 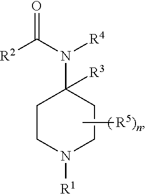
wherein
R¹ is H or C₁₋₆alkyl, optional substituted one or more times by OH, CO₂C₁₋₆alkyl, aryl, C₃₋₁₀cycloalkyl, C₂₋₁₀heterocyclyl, or C₁₋₁₀heteroaryl;
R² is C₁₋₆alkyl, C₂₋₄alkenyl, C₃₋₁₀cycloalkyl or C₁₋₁₀heteroaryl; wherein R² may be substituted by aryl, OC₁₋₆alkyl;
R³ is H, CO₂C₁₋₆alkyl, C₁₋₆alkyl, aryl, wherein R³ may be substituted by OC₁₋₆alkyl;
R⁴ is aryl or C₁₋₁₀heteroaryl, wherein R⁴ substituted one or more times by F;
R⁵ is in each case independently selected from C₁₋₆alkyl, C₂₋₄alkenyl, and F; and
n is 0, 1, or 2.

18. The method according to claim 16, wherein the fentanyl analog is sufentanil, alfentanil, remifentanil, and carfentanil, furanylfentanyl, 4-fluorobutyrylfentanyl, 4-methoxyburtyrylfentanyl, acrylfentanyl, 4-chloroisobutyryfentanyl (4Cl-iBF), 4-fluoroisobutyrfentanyl (4F-iBF), tetrahydrofuranfentanyl (THF-F), cyclopentylfentanyl, AH-7921, U-47700, MT-45, AH-7921, U-47700, AH-7921, or a combination thereof.

19. The method according to any of claims 1-3, wherein the opioid use disorder is fentanyl overdose, fentanyl analog overdose, or a combination thereof.

20. The method according to any of claims 1-3, wherein the opioid use disorder is opioid overdose with concomitant use of alcohol, barbiturates, xylazine, benzodiazepines, cocaine, amphetamines, gamma-hydroxy butyrate, PCP, ketamine, or a combination thereof.

21. The method according to any of claims 1-3, wherein the opioid use disorder is opioid addiction.

22. A method for supporting opioid cessation in a subject in need thereof, comprising administering to the subject an effective amount of methocinnamox, or a pharmaceutically acceptable salt thereof, in combination with at least one additional opioid receptor modulator.

23. A method of treating an alcohol use disorder in a subject, comprising administering to the subject in need thereof an effective amount of methocinnamox, or a pharmaceutically acceptable salt thereof, in combination with at least one additional opioid receptor modulator.

24. The method according to claim 23, wherein the alcohol use disorder is alcohol dependence.

25. The method according to claim 23, wherein the alcohol use disorder is alcohol detoxification.

26. The method according to any of claims 23-25, wherein the method is for reducing alcohol cravings.

27. The method according to claim 23, wherein the method is for treating or preventing alcohol overdose.

28. The method according to claim 23, wherein the method is for reducing or preventing alcohol use disorder in an at-risk patient.

29. The method according to claim 23, wherein the at-risk patient is a trauma-survivor.

30. A method for reducing alcohol consumption in a subject, comprising administering to the subject in need thereof an effective amount of methocinnamox, or a pharmaceutically acceptable salt thereof, in combination with at least one additional opioid receptor modulator.

31. A method for promoting alcohol use cessation in a subject, comprising administering to the subject in need thereof an effective amount of methocinnamox, or a pharmaceutically acceptable salt thereof, in combination with at least one additional opioid receptor modulator.

32. The method according to claim 30 or 31, further comprising administering an alcohol dehydrogenase inhibitor, NMDA antagonist, anticonvulsant, antidepressant, other serotonergic agent, or combination thereof like.

33. The method according to claim 32, wherein the alcohol dehydrogenase inhibitor is disulfiram, the NMDA antagonist is acamprosate, the anticonvulsant is gabapentin, pregabalin, or topiramate.

34. The method according to claim 30 or 31, wherein the additional opioid receptor modulator is naltrexone, naloxone, nalmefene, diprenorphine, nalorphine, nalorphine dinicotinate, levallorphan, samidorphan, nalodiene, buprenorphine, dezocine, eptazocine, butorphanol, levorphanol, nalbuphine, pentazocine, phenazocine, cyprodime, naltrindole, norbinaltorphimine, salts thereof, or a combination thereof.

35. The method according to claim 34, wherein the additional opioid receptor modulator is naltrexone, or a pharmaceutically acceptable salt thereof.

36. The method according to claim 34, wherein the additional opioid receptor modulator is naltrexone HCl.

37. The method according to claim 34, wherein the additional opioid receptor modulator is naloxone, or a pharmaceutically acceptable salt thereof.

38. The method according to claim 34, wherein the additional opioid receptor modulator is naloxone HCl.

39. The method according to any of claims 1-38, wherein the methocinnamox, or pharmaceutically acceptable salt thereof, is administered at a dose of 0.1-10 mg/kg, 0.1-8 mg/kg, 0.1-6 mg/kg, 0.1-5 mg/kg, 0.1-4 mg/kg, 0.1-3 mg/kg, 0.1-2 mg/kg, 0.1-1 mg/kg, 0.5-1.5 mg/kg, 1-2 mg/kg, 1.5-2.5 mg/kg, 2-5 mg/kg, 2.5-7.5 mg/kg, or 5-10 mg/kg (calculated as the free base).

40. The method according to any of claims 1-38, wherein the methocinnamox, or pharmaceutically acceptable salt thereof, is administered at a dose of 0.5-500 mg, 0.5-250 mg, 0.5-100 mg, 0.5-50 mg, 0.5-25 mg, 0.5-20 mg, 0.5-15 mg, 0.5-10 mg, 0.5-5 mg, 0.5-2.5 mg, 1-2.5 mg, 1-5 mg, 1-10 mg, 2.5-7.5 mg, 5-15 mg, 10-20 mg, 15-25 mg, 20-30 mg, 25-50 mg, 25-75 mg, 50-100 mg, 100-250 mg, or 250-500 mg, wherein the amount is measured as the free base equivalent.

41. The method according to any of claims 1-40, wherein the additional opioid receptor modulator is administered at a dose of 0.01-3 mg/kg, 0.01-2 mg/kg, 0.01-1 mg/kg, 0.05-1 mg/kg, 0.1-1 mg/kg, 0.1-0.5 mg/kg, 0.25-0.75 mg/kg, 0.5-1 mg/kg, 0.01-0.1 mg/kg, 0.05-0.1 mg/kg, 0.025-0.075 mg/kg, 0.01-0.05 mg/kg, or 0.075-0.125 mg/kg, wherein the amount is measured as the free base equivalent.

42. The method according to any of claims 1-40, wherein the additional opioid receptor modulator is administered at a dose of 0.5-100 mg, 0.5-50 mg, 0.5-25 mg, 0.5-20 mg, 0.5-15 mg, 0.5-10 mg, 0.5-5 mg, 0.5-2.5 mg, 1-2.5 mg, 1-5 mg, 1-10 mg, 2.5-7.5 mg, 5-15 mg, 10-20 mg, 15-25 mg, 20-30 mg, 25-50 mg, 25-75 mg, or 50-100 mg, wherein the amount is measured as the free base equivalent.

43. The method according to any of claims 1-42, wherein the methocinnamox and the additional opioid receptor modulators are administered in the same event dosing event.

44. The method according to any of claims 1-42, wherein the methocinnamox and the additional opioid receptor modulators are administered within a period of 4 hours, 3 hours, 2 hours, 1 hour, 30 minutes, 15 minutes, 10 minutes, 5 minutes, or 2 minutes.

45. The method according to any of claims 1-42, wherein the methocinnamox is administered before the additional opioid receptor modulator.

46. The method according to any of claims 1-42, wherein the methocinnamox is administered after the additional opioid receptor modulator.

47. The method according to any of claims 1-46, wherein the methocinnamox is administered enterally, parenterally, intranasally, vaginally, by inhalation, or a combination thereof.

48. The method according to any of claims 1-47, wherein the methocinnamox is administered enterally by oral administration, sublingual administration, buccal administration, rectal administration, or a combination thereof.

49. The method according to any of claims 1-47, wherein the methocinnamox is administered parenterally by intramuscular injection, intravenous injection, subcutaneous injection, or a combination thereof.

50. The method according to any of claims 1-47, wherein the methocinnamox is administered intranasally.

51. The method according to any of claims 1-47, wherein the methocinnamox is administered by inhalation.

52. The method according to any of claims 1-52, wherein the additional opioid receptor modulator is administered enterally, parenterally, intranasally, vaginally, by inhalation, or a combination thereof.

53. The method according to any of claims 1-52, wherein the additional opioid receptor modulator is administered enterally by oral administration, sublingual administration, buccal administration, rectal administration, or a combination thereof.

54. The method according to any of claims 1-52, wherein the additional opioid receptor modulator is administered parenterally by intramuscular injection, intravenous injection, subcutaneous injection, or a combination thereof.

55. The method according to any of claims 1-52, wherein the additional opioid receptor modulator is administered intranasally.

56. The method according to any of claims 1-52, wherein the additional opioid receptor modulator is administered by inhalation.

57. The method according to any preceding claim, wherein the methocinnamox and additional opioid receptor modulator are administered in the same unit dose.

58. A pharmaceutical composition, comprising methocinnamox, or a pharmaceutically acceptable salt thereof, and at least one additional opioid receptor modulator.

59. The pharmaceutical composition according to claim 58, wherein the additional opioid receptor modulator is naltrexone, naloxone, nalmefene, diprenorphine, nalorphine, nalorphine dinicotinate, levallorphan, samidorphan, nalodiene, buprenorphine, dezocine, eptazocine, butorphanol, levorphanol, nalbuphine, pentazocine, phenazocine, cyprodime, naltrindole, norbinaltorphiminc, salts thereof, or a combination thereof.

60. The pharmaceutical composition according to claim 58 or 59, wherein the additional opioid receptor modulator is naltrexone, or a pharmaceutically acceptable salt thereof.

61. The pharmaceutical composition according to any of claims 58-60, wherein the additional opioid receptor modulator is naltrexone HCl.

62. The pharmaceutical composition according to any of claims 58-61, wherein the additional opioid receptor modulator is naloxone, or a pharmaceutically acceptable salt thereof.

63. The pharmaceutical composition according to any of claims 58-62, wherein the additional opioid receptor modulator is naloxone HCl.

64. The pharmaceutical composition according to any of claims 58-63, comprising methocinnamox, or pharmaceutically acceptable salt thereof, in an amount of 0.5-500 mg, 0.5-250 mg, 0.5-100 mg, 0.5-50 mg, 0.5-25 mg, 0.5-20 mg, 0.5-15 mg, 0.5-10 mg, 0.5-5 mg, 0.5-2.5 mg, 1-2.5 mg, 1-5 mg, 1-10 mg, 2.5-7.5 mg, 5-15 mg, 10-20 mg, 15-25 mg, 20-30 mg, 25-50 mg, 25-75 mg, 50-100 mg, 100-250 mg, or 250-500 mg, wherein the amount is measured as the free base equivalent.

65. The pharmaceutical composition according to any of claims 58-64, comprising the additional opioid receptor modulator in an amount of 0.5-100 mg, 0.5-50 mg, 0.5-25 mg, 0.5-20 mg, 0.5-15 mg, 0.5-10 mg, 0.5-5 mg, 0.5-2.5 mg, 1-2.5 mg, 1-5 mg, 1-10 mg, 2.5-7.5 mg, 5-15 mg, 10-20 mg, 15-25 mg, 20-30 mg, 25-50 mg, 25-75 mg, or 50-100 mg, wherein the amount is measured as the free base equivalent.

66. A kit for the treatment of an opioid use disorder, comprising methocinnamox, or a pharmaceutically acceptable salt thereof, and at least one additional opioid receptor modulator.

67. The kit according to claim 66, wherein the additional opioid receptor modulator is naltrexone, naloxone, nalmefene, diprenorphine, nalorphine, nalorphine dinicotinate, levallorphan, samidorphan, nalodiene, buprenorphine, dezocine, eptazocine, butorphanol, levorphanol, nalbuphine, pentazocine, phenazocine, cyprodime, naltrindole, norbinaltorphimine, salts thereof, or a combination thereof.

68. The kit according to claim 66 or 67, wherein the additional opioid receptor modulator is naltrexone, or a pharmaceutically acceptable salt thereof.

69. The kit according to any of claims 66-68, wherein the additional opioid receptor modulator is naltrexone HCl.

70. The kit according to any of claims 66-69, wherein the additional opioid receptor modulator is naloxone, or a pharmaceutically acceptable salt thereof.

71. The kit according to any of claims 66-70, wherein the additional opioid receptor modulator is naloxone HCl.

72. The kit according to any of claims 66-71, wherein the methocinnamox and additional opioid receptor modulator are in separate unit dosage forms.

73. The kit according to any of claims 66-71, wherein the methocinnamox and additional opioid receptor modulator are in the same unit dosage form.
